(19) United States
(12) Patent Application Publication
(10) Pub. No.: US 2010/0179521 A1
(43) Pub. Date: Jul. 15, 2010

(54) MULTIPURPOSE INTENSE PULSED LIGHT SYSTEM

(76) Inventor: Shahriar Ghaffari, Austin, TX (US)

Correspondence Address: Mehdi Arabshahi, 11509 North Lamar Blvd., Austin, TX, 78753, US

(21) Appl. No.: 12/686,904

(22) Filed: Jan. 13, 2010

Related U.S. Application Data
(60) Provisional application No. 61/204,888, filed on Jan. 13, 2009.

(51) Int. Cl. A61B 18/18

(52) U.S. Cl. 606/9

(57) ABSTRACT

Apparatus and methods disclosed herein operate to produce high peak power light output, to deliver the light output to a portion of skin in a dermatological setting, to deliver a flow of cool gas or air to the skin for cooling purposes, to monitor skin temperature during the delivery of light to the portion of skin, and to adjust the light power in real time such as to enhance the tradeoff between treatment efficacy and skin damage.

### PRIORITY CLAIMS

This disclosure claims the benefit of the filing date of Provisional Patent Application Ser. No. 61/204,888, filed on Jan. 13, 2009, and titled “Multipurpose Intense Pulse Light Device.”

### TECHNICAL FIELD

Various embodiments described herein relate to medical devices and techniques including apparatus and methods associated with electromagnetic irradiation of tissue in clinical dermatology using an intense pulsed-light (IPL) radiation source.

### BACKGROUND INFORMATION

Lamps, lasers, and other sources of electromagnetic radiation are increasingly being used for skin treatments. The absorption of light energy by a target within skin and the resulting coagulation of the target can produce desired clinical results. In particular, light has been utilized to remove unwanted hair, eliminate leg veins, remove tattoos, and remove or reduce acne vulgaris and skin lesions. Non-laser pulsed light treatments use intense pulsed light (IPL) to coagulate a target cellular structure using a photo selective process. The skin is protected from thermal injury as it is allowed to cool between and/or during pulses of light while high temperature is induced in the target.

One of the advantages of incoherent light based systems over laser systems is that it is possible to efficiently obtain a suitable light energy flux over a larger area. Quickly and uniformly exposing a large treatment area reduces treatment time. Treatment of a particular target tissue type may be enhanced by utilizing a suitable set of wavelengths. A single wide-band IPL device may be adapted for a selected treatment type by changing bandpass filters and thereby selecting the indicated set of wavelengths.

Limitations exist as to acceptable radiation fluence levels used for various dermatological treatments. Excessive levels may cause damage to the epidermis or epidermis-dermis junction. Consequently, epidermal cooling may be used when high level irradiation is required for successful clinical treatment. Currently, protection of the epidermis is achieved by cooling the skin surface by a cold sapphire window or spraying the skin with a short cryogen spurt.

The U.S. Pat. No. 5,226,907, to N. Tankovich, HAIR REMOVAL DEVICE AND METHOD, describes a hair removal method based on darkening the hair and hair color to enhance absorption of light by the follicles, to improve efficacy. The problems with this method are there is no skin cooling to protect the epidermis and to reduce discomfort during treatments.

The U.S. Pat. No. 5,683,380 to Eckhouse et al., METHOD AND APPARATUS FOR DEPLIATION USING PULSED ELECTROMAGNETIC RADIATION, describes a hair removal apparatus based on one flashlamp. In this invention a gel is disposed on the surface of the tissue and the window is in contact with the gel. Contact cooling reduces the speed that the handpiece can move across the skin thereby limiting the maximum speed of treatments. Furthermore; the lack of multiple flashlamps severely limits the spot size to less than 10 sq. cm per pulse of light that the Eckhouse device is able to treat at the proper fluence levels.

The U.S. Pat. No. 6,511,475 to Altshuler et al, HEAD FOR DERMATOLOGY TREATMENT, describes the use of continuous wave radiation as apposed to a pulsed light system. This device is not capable of producing high peak powers to achieve sufficient peak temperature in a short time duration, which severely limits its use in a wide variety of skin conditions.

The U.S. Pat. No. 7,097,639 B1 to Almeida, DUAL FILTER MULTIPLE PULSE PHOTO-DERMATOLOGICAL DEVICE . . . , describes the optimum fixed specification wavelength distribution pattern for the treatment of various skin conditions by adjusting the intensity of light and delay between pulses. Skin cooling for the treatment area is not recommended. One deficiency of such device is that the generated light is not distributed evenly. Further, the lack of skin cooling severely limits the use of the device for dark skin, and the proposed algorithm is not adaptable for different skin types in any given treatment session.

### BRIEF DESCRIPTION OF THE DRAWINGS

FIG. 1 is a cross-sectional view of a skin treatment system according to various example embodiments.

FIG. 2 is a perspective view of a portion of a handpiece associated with a skin treatment system according to various example embodiments.

FIG. 3 is a partially cut-away side perspective view of the handpiece according to various example embodiments.

FIG. 4a is a partially cut-away side view of the handpiece according to various example embodiments.

FIGS. 4b and 4c are Four-Lamps and Six-Lamps configuration within handpiece.

FIG. 5 is a partially cut-away side perspective view of the handpiece according to various example embodiments.

FIG. 6 is a flow diagram according to various example embodiments.

FIG. 7 is a block diagram of a controller associated with the skin treatment system according to various example embodiments.

### DETAILED DESCRIPTION

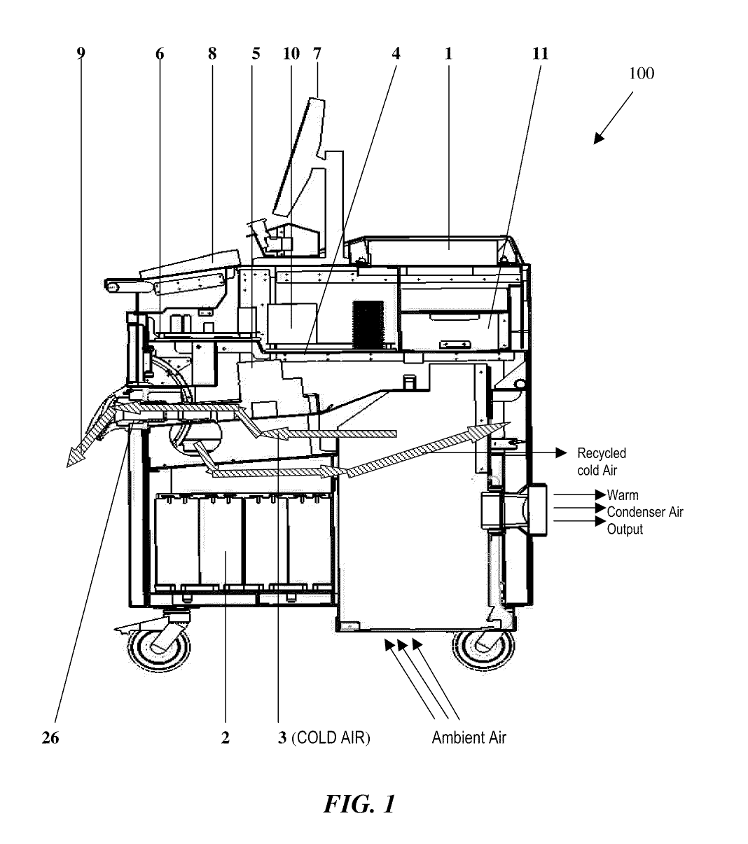

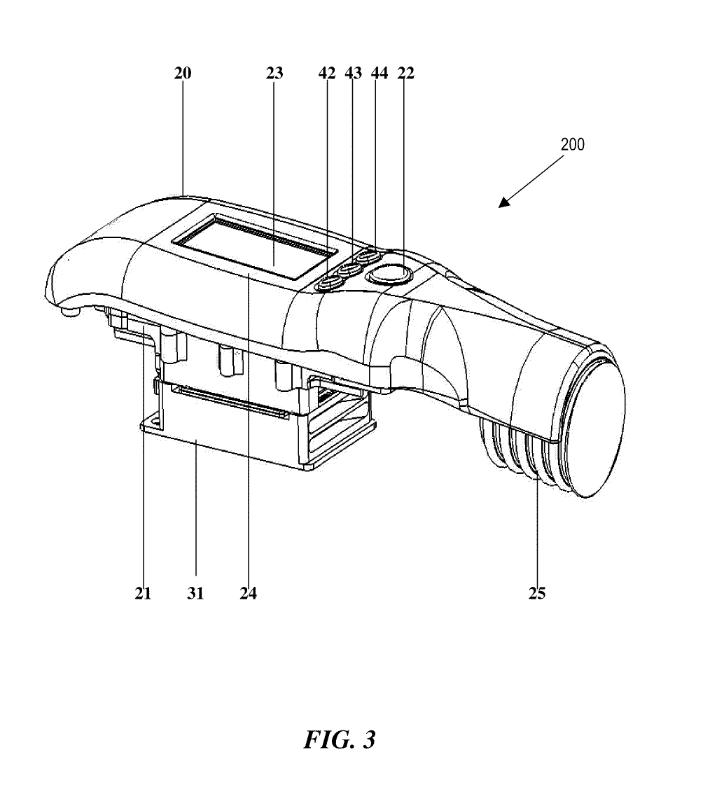

FIG. 1 is a cross-sectional view of a skin treatment system 100 according to various example embodiments. Embodiments herein may be suitable for dermatological skin treatment including but not limited to skin rejuvenation and/or to the removal of unwanted hair, tattoos, acne, and vascular and pigmentation lesions. In some embodiments, the skin treatment system 100 includes a computer 1, energy storage capacitor banks 2, a pulse cooling device 3, a main motherboard 4, and a handpiece adapter 26. Computer 1 manages communications with the handpiece 20 (shown in FIG. 3) and main motherboard 4. Such communications are effected via Universal Serial Bus (USB) interface and communication software in some embodiments.

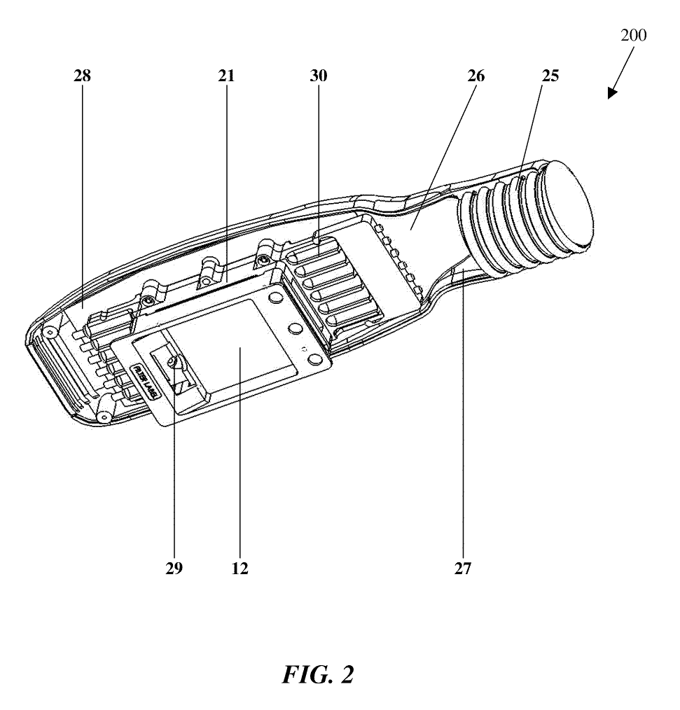

FIG. 2 is a perspective view of a portion of a handpiece 200 associated with the skin treatment system 100 according to various example embodiments. The handpiece 200 includes a flashlamp array 30 to provide light radiation for treatment. Light emitted from the flashlamp array 30 may be filtered by an internal filter 12 to output wavelengths from 420 to 1100 nanometers (nm) at maximum fluences in excess of 50 Joules/cm². An array of falshlamp can produce 50 Joules/cm² into large spot size of 15 cm² or more. The choice of 3 or more falshlamps produce fluences in the 50 Joules/cm² in large spot sizes of 15 cm² or more.

An infrared sensor 29 within the housing 21 of the handpiece 200 may be positioned at an angle to facilitate skin temperature measurements. The apertures and other components of the infrared sensor 29 are described in detail further below. In FIG. 2, the outputs of thermistor 26 (not shown in FIG. 2), measuring temperature of incoming air, thermistor 27 (not shown in FIG. 2), measuring temperature of the air directed for skin cooling, thermistor 28 (not shown in FIG. 2), measuring outgoing air temperature, and infrared sensor 29, measuring skin temperature, are measured by the main computer 1 to form a baseline for treatment. The main computer 1 uses the temperature information to control the treatment parameters.

FIG. 3 is a partially cut-away side perspective view of the handpiece 200 according to various example embodiments. The handpiece 200 may include a handpiece cover 20, an optical housing 21, a display 23, a trigger switch 22, and an automatically recognizable external filter 31. The handpiece 200 is activated by the trigger switch 22. The parameters can be modified by push buttons 42, 43, and 44. Apparatus 20 includes a handpiece circuit 24 (not shown in FIG. 3) that controls and ssynchronizes communications with the main computer 1 and monitors sensors utilized in the handpiece 200. Apparatus 20 includes a display 23 for providing useful information including but not limited to skin type setting, skin temperature, filter type, and fluence levels being delivered to the skin. The control electronics, flashlamp power cables, and cold air are combined and connected to the handpiece 20 via an umbilical hose 25.

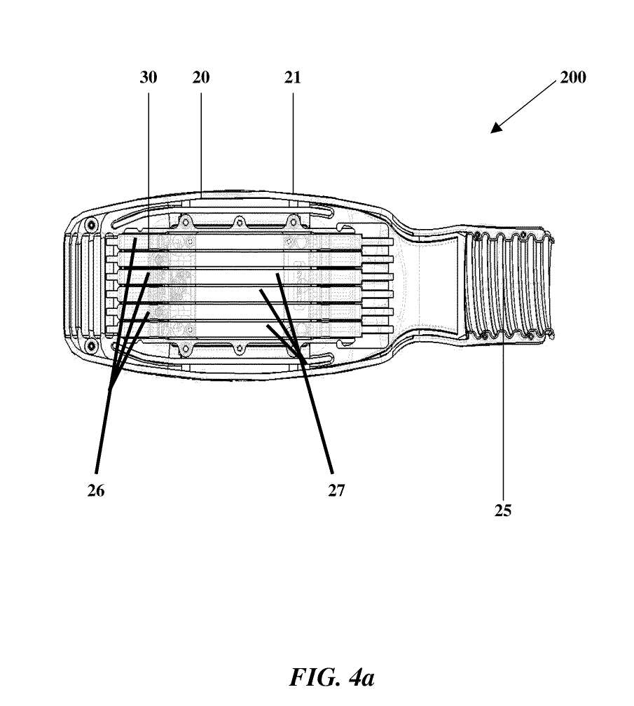

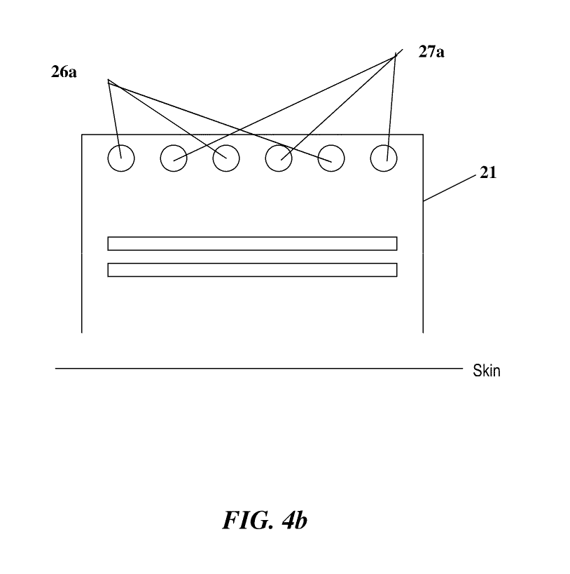

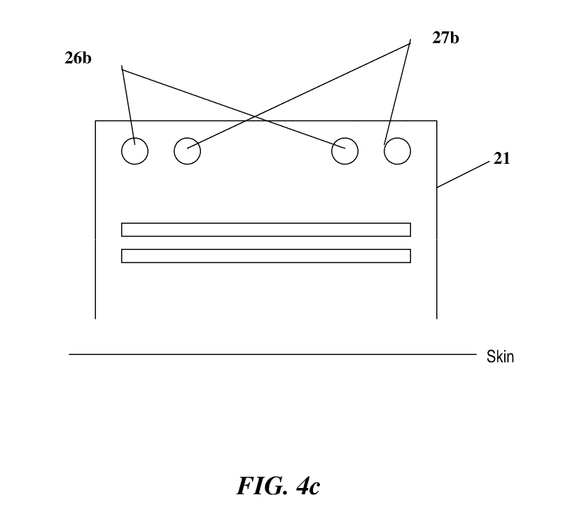

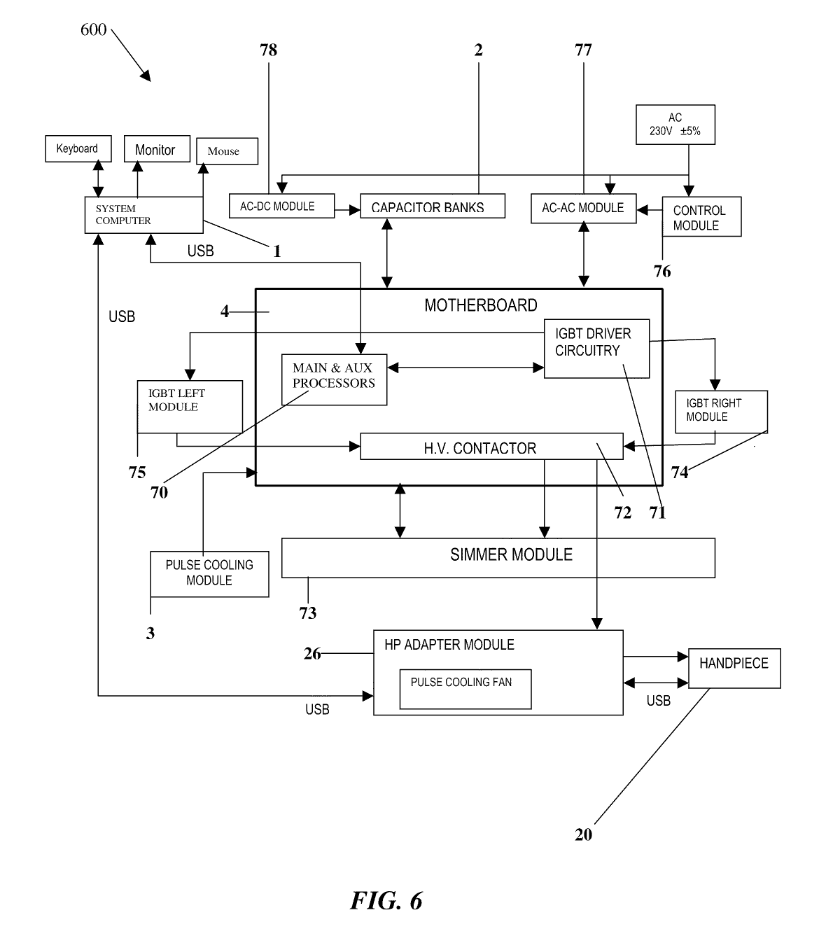

FIG. 4a is a partially cut-away side view of the handpiece 200 according to various example embodiments. The handpiece 200 includes a housing 21 and an array of flashlamps 30 to provide light radiation for treatment. The flashlamp array 30 includes flashlamp set 26 and flashlamp set 27, the latter controlled by two separate energy delivery unit 2 as shown in FIG. 6, module 74 and module 75.

FIGS. 4b and 4c are Six-Lamp and Four-Lamp configurations within optical housing 21.

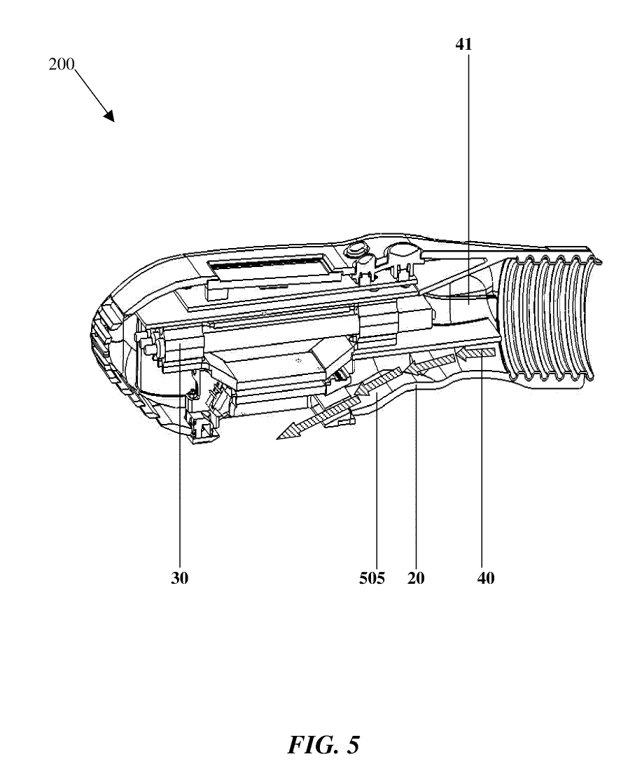

FIG. 5 is a partially cut-away side perspective view of the handpiece 200 according to various example embodiments. The handpiece cover 20, as discussed above, includes an airflow channel 505 to direct cool air 40 to the skin and airflow channel 41 to cool the flashlamp assembly 30.

FIG. 6 is a block flow diagram illustrating a method 600 according to various example embodiments. The method 600 includes performing parallel processing of the information for handpiece 20, monitoring and controlling energy stored in capacitor bank 2, and dumping energy into the bank of flashlamps via IGBT's 74 and 75. The power supply 78 converts the 220 volts alternating current (AC) into the capacitor bank direct current (DC) voltage in the range of 160-750 volts. The high voltage contactor 72 isolates the system from the ignition voltages during flashlamp start up process. Pulse cooling fan inside the HP adapter module 26, utilizing a high power blower, is controlled by the main computer 1. Different skin types change the algorithm to alternate the different flashlamp banks. The higher the skin type index result into lower initial peak powers ramping to the highest peak power at the end of the alternating pulse series. The alternating control of flashlamp bank produces decaying, flat or increasing rate of peak power pulse series when a series of light pulses are delivered to skin.

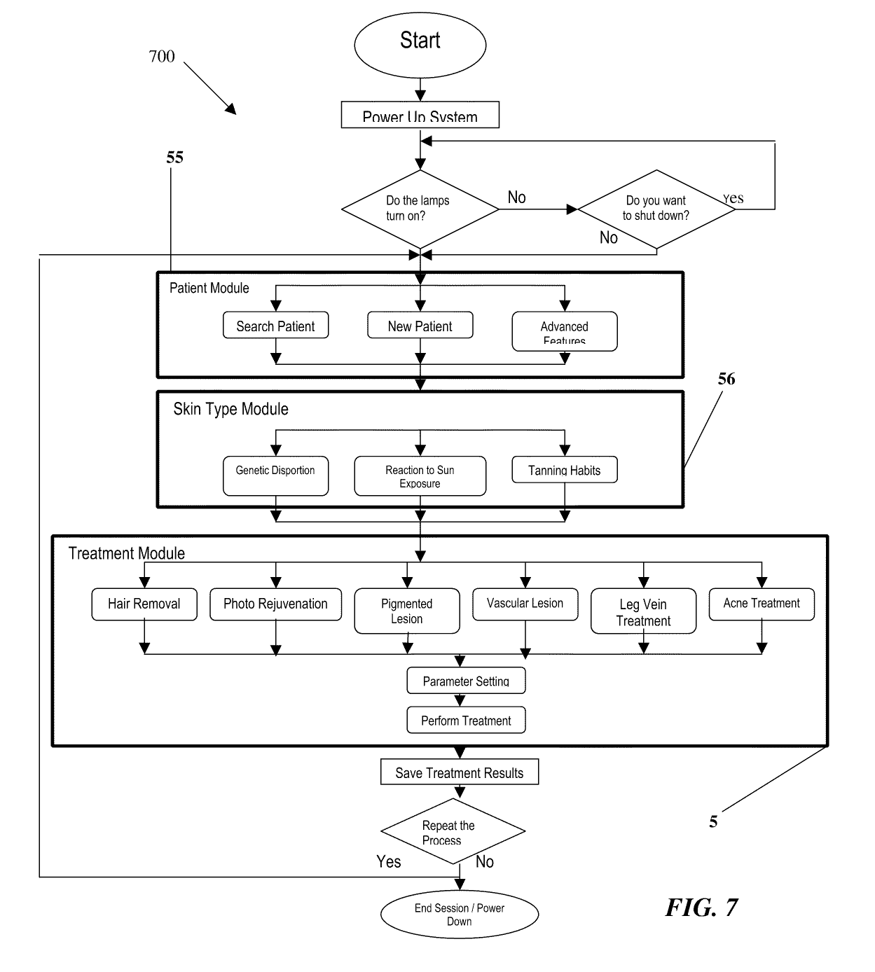

FIG. 7 is a flow diagram of the computer software utilized in this invention. FIG. 7 includes a high level Graphical User Interface (GUI), which interacts with the operator. The Patient Module 55 in FIG. 7 is responsible for recording, maintaining and updating patient parameters used during treatments with corresponding pictures, notes and voice recordings. The Skin Type Module 56 in FIG. 7 determines the patient skin type based on his/her medical background and skin reaction information. The Treatment Module 57 in FIG. 7 sets parameters, interfaces with Microcontrollers, and activates the handpiece based on the parameters appropriate for that treatment indication.

The components and modules described herein may include hardware circuitry, optical components, multi-processor circuits, memory circuits, software program modules and objects encoded in a computer-readable medium and capable of being executed by a processor (but excluding non-functional descriptive matter), firmware, and combinations thereof, as desired by the architect of the skin treatment system 100 and as appropriate for particular implementations of various embodiments.

The apparatus, systems, and methods of various embodiments may be useful in applications other than the skin treatment system 100. Thus, various embodiments of the invention are not to be so limited. The illustration of the skin treatment system 100 is intended to provide a general understanding of the structure of various embodiments. It is not intended to serve as a complete description of all the elements and features of apparatus and systems that might make use of the structures described herein.

The novel apparatus and systems of various embodiments may comprise or be incorporated into a variety of electronic systems, such as televisions, cellular telephones, personal computers (e.g., laptop computers, desktop computers, handheld computers, tablet computers, etc.), workstations, radios, video players, audio players (e.g., MP3 (Motion Picture Experts Group, Audio Layer 3) players), vehicles, medical devices (e.g., heart monitor, blood pressure monitor, etc.), set top boxes, and others. Some embodiments may include a number of methods.

It is noted that the activities described herein may be executed in an order other than the order described. The various activities described with respect to the methods identified herein may also be executed in repetitive, serial, and/or parallel fashion.

A software program may be launched from a computer-readable medium in a computer-based system to execute functions defined in the software program. Various programming languages may be employed to create software programs designed to implement and perform the methods disclosed herein. The programs may be structured in an object-oriented format using an object-oriented language such as Labview, Java or C++. Alternatively, the programs may be structured in a procedure-oriented format using a procedural language, such as assembly or C. The software components may communicate using well-known mechanisms, including application program interfaces, inter-process communication techniques, and remote procedure calls, among others. The teachings of various embodiments are not limited to any particular programming language or environment.

The apparatus, systems, and methods described herein may operate to irradiate skin at predetermined wavelength spectra and to cool the skin during a predetermined time interval. Skin irradiation and cooling levels may be coordinated while measuring skin temperature to increase patient comfort levels.

By way of illustration and not of limitation, the accompanying figures show specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This Detailed Description, therefore, is not to be taken in a limiting sense. The breadth of various embodiments is defined by the appended claims and the full range of equivalents to which such claims are entitled.

Such embodiments of the inventive subject matter may be referred to herein individually or collectively by the term “invention” merely for convenience and without intending to voluntarily limit this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments and other embodiments not specifically described herein will be apparent to those of skill in the art upon reviewing the above description.

The Abstract of the Disclosure is provided to comply with 37 C.F.R. §1.72(b) requiring an abstract that will allow the reader to quickly ascertain the nature of the technical disclosure. It is submitted with the understanding that it will not be used to interpret or limit the scope or meaning of the claims. In the preceding Detailed Description, various features are grouped together in a single embodiment for the purpose of streamlining the disclosure. This method of disclosure is not to be interpreted to require more features than are expressly recited in each claim. Rather, inventive subject matter may be found in less than all features of a single disclosed embodiment. Thus the following claims are hereby incorporated into the Detailed Description, with each claim standing on its own as a separate embodiment.

What is claimed is:

1. A dermatological apparatus, comprising:
an array of flashlamps to produce high peak power light output, said array of flashlamps positioned proximate to a reflector;
means to provide controlled high current for said flashlamp array;
means to collect and deliver light to a portion of skin;
a cooling system to simultaneously cool a treatment area before, during, and after delivery of light;
a monitoring system to allow monitoring the output power of said flashlamps as light interacts with a treatment site on skin.

2. The apparatus of claim 1, wherein said flashlamp array is comprised of at least three flashlamps.

3. The apparatus of claim 1, wherein said delivery of light comprises a spot size larger than 15 square cm.

4. The apparatus of claim 1, further including:
at least one infrared sensor to detect skin temperature.

5. An apparatus for treating skin comprising:
a handpiece including at least one flashlamp and a reflector to form a flashlamp assembly;
a cooling system to provide cooled gas or air to cool said handpiece;
a means to direct a flow of said cooled gas or air to a site on a portion of skin;
a means to cool the flashlamp assembly with a portion of said cooled gas or air;
a means to display and adjust treatment parameters;
a means to deliver high energy light to said portion of skin simultaneously with said flow of gas or air across said skin.

6. The apparatus of claim 5, further including:
a computer to control said flow of gas or air.

7. The apparatus of claim 5, further including;
a computer to monitor at least one of high energy output or skin temperature.

8. An apparatus for treating skin comprising:
a power supply;
a handpiece incorporating an array of flashlamps;
pulsed gas cooling of a treatment site on a portion of skin;
a computerized monitoring and feedback system to control treatment settings based upon skin type.

9. The apparatus of claim 8, further including:
a mounting area within said handpiece to accept the monitoring and feedback signals.

10. The apparatus of claim 8, further including:
a treatment parameter input module to accept and display treatment parameters associated with said feedback system.

11. An apparatus according to claim 8, wherein said skin type based algorithm comprises means for user selecting and controlling the peak intensity by alternating banks of flashlamps.

12. An apparatus according to claim 11, further comprising a power supply that is adapted to control the current to each said banks of flashlamps.

13. An apparatus according to claim 8, wherein said pulsed cooling system is comprised of a high power blower with a variable computer controlled output air flow.

14. An apparatus for treating skin and having a light source for emission of treatment light directed onto a site on skin, a cooling means to cool skin, a monitoring means for detection of skin temperature and a display for displaying treatment parameters based on said monitoring means.

15. An apparatus according to claim 14, wherein said monitoring means for detection of skin temperature is comprised of at least one infrared sensor.

16. An apparatus according to claim 14, wherein said monitoring means displays the actual temperature of skin to be treated before, during and/or after treatment.

17. An assembly comprising at least one bank of multiple flashlamps proximate to a reflector positioned in a plane;
Wherein each bank of flashlamps is placed at a predetermined distance from said reflector, to produce uniform light distribution on a target.

18. An apparatus according to claim 17, wherein said bank of flashlamp array is comprised of at least two flashlamps.

19. An assembly comprising at least two banks of flashlamps with each bank comprising at least two flashlamps;
Wherein each said bank produces a uniform light distribution on a target;
Whereby said bank of flashlamps is controlled to activate in coordination with other bank(s) to control the peak output light intensity.

20. An assembly comprising an air conditioning system within an enclosure, said air conditioning system equipped with an air inlet, placed inside said assembly;
cooling said air inside said enclosure;
recycling a portion of said cooled air back to said air inlet;
delivering a portion of said cooled air to a handpiece;
controlling a flow of said delivered air by a computer.
